(19) United States
(12) Patent Application Publication
(10) Pub. No.: US 2024/0168002 A1
(43) Pub. Date: May 23, 2024

(54) INTERACTIVE CORRELATION AND PREDICTION OF SOURCE ROCK ORGANOFACIES, OIL FAMILIES AND RESERVOIR ALTERATION USING MACHINE LEARNING

(71) Applicant: SAUDI ARABIAN OIL COMPANY, Dhahran (SA)

(72) Inventors: Assad Hadi Ghazwani, Muharraq (BH); Maram Saif, Dhahran (SA); Khaled Arouri, Dhahran (SA)

(73) Assignee: SAUDI ARABIAN OIL COMPANY, Dhahran (SA)

(21) Appl. No.: 17/990,542

(22) Filed: Nov. 18, 2022

(51) Int. Cl. G01N 33/24; G01N 30/72

(52) U.S. Cl. CPC G01N 33/241; G01N 30/7206

(57) ABSTRACT

Systems and methods are disclosed. The method includes obtaining geochemical data and geological data for a number of oil samples and training a machine learning network using the geochemical data and the geological data. Each oil sample includes hydrocarbon molecules and the geochemical data includes abundances of the hydrocarbon molecules. The method further includes obtaining a new oil sample from a subterranean region of interest and determining new geochemical data for the new oil sample using gas chromatography. The method still further includes predicting new geological data for the new oil sample by inputting the new geochemical data into the trained machine learning network.

### BACKGROUND

An oil field may contain multiple hydrocarbon reservoirs generated by various source rocks. As an oil field is explored, appraised, and developed to produce hydrocarbons from the hydrocarbon reservoirs to the surface, oil samples may be collected. Oil samples may be analyzed in a laboratory to generate a vast amount of geochemical data. In turn, the geochemical data may be interpreted to determine geological data, such as origin data, depositional environment data, organofacies data, oil family data, and alteration mechanism data, for each oil sample. However, traditionally, the vast geochemical data may require manual organization and interpretation by a geochemist to extract useful geological data. Following the successful organization and interpretation of geochemical data, the geological data may be used to determine an oil field management plan.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method. The method includes obtaining geochemical data and geological data for a number of oil samples and training a machine learning network using the geochemical data and the geological data. Each oil sample includes hydrocarbon molecules and the geochemical data includes abundances of the hydrocarbon molecules. The method further includes obtaining a new oil sample from a subterranean region of interest and determining new geochemical data for the new oil sample using gas chromatography. The method still further includes predicting new geological data for the new oil sample by inputting the new geochemical data into the trained machine learning network.

In general, in one aspect, embodiments relate to a non-transitory computer-readable memory having computer-executable instructions stored thereon that are executable by a computer processor. The computer-executable instructions cause the computer processor to perform steps that include obtaining geochemical data and geological data for a number of oil samples and training a machine learning network using the geochemical data and the geological data. Each oil sample includes hydrocarbon molecules and the geochemical data includes abundances of the hydrocarbon molecules. The steps further include receiving new geochemical data for a new oil sample from a subterranean region of interest. The steps further still include predicting new geological data for the new oil sample by inputting the new geochemical data into the trained machine learning network.

In general, in one aspect, embodiments relate to a system. The system includes a gas chromatography system configured to determine new geochemical data for a new oil sample. The system further includes a computer processor configured to obtain geochemical data and geological data for a number of oil samples and train a machine learning network using the geochemical data and the geological data. Each oil sample includes hydrocarbon molecules and the geochemical data includes abundances of the hydrocarbon molecules. The computer processor is further configured to receive the new geochemical data for the new oil sample. The computer processor is still further configured to predict new geological data for the new oil sample by inputting the new geochemical data into the trained machine learning network.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 illustrates a subterranean region of interest in accordance with one or more embodiments.

FIG. 2 illustrates a production system in accordance with one or more embodiments.

FIG. 3 shows a chromatogram in accordance with one or more embodiments.

FIG. 4 shows a plot in accordance with one or more embodiments.

FIGS. 5A and 5B show star diagrams in accordance with one or more embodiments.

FIG. 6 illustrates a random forest algorithm in accordance with one or more embodiments.

FIG. 7 illustrates a neural network in accordance with one or more embodiments.

FIG. 8 illustrates convolution in accordance with one or more embodiments.

FIG. 9 shows a workflow in accordance with one or more embodiments.

FIG. 10 shows a flowchart in accordance with one or more embodiments.

FIG. 11 shows a geological map in accordance with one or more embodiments.

FIG. 12 depicts systems in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a geological map” includes reference to one or more of such maps.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowchart may be omitted, repeated, and/or performed in a different order than the order shown. Accordingly, the scope disclosed herein should not be considered limited to the specific arrangement of steps shown in the flowchart.

Although multiple dependent claims are not introduced, it would be apparent to one of ordinary skill that the subject matter of the dependent claims of one or more embodiments may be combined with other dependent claims.

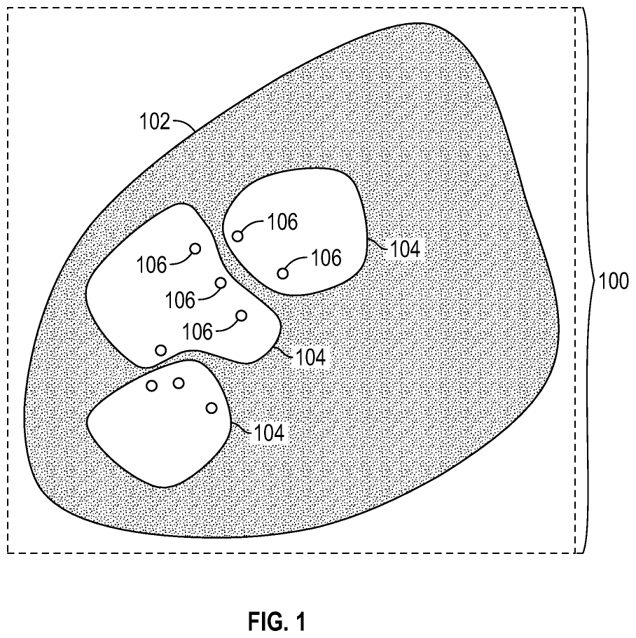

In the following description of FIGS. 1-12, any component described regarding a figure, in various embodiments disclosed herein, may be equivalent to one or more like-named components described regarding any other figure. For brevity, descriptions of these components will not be repeated regarding each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described regarding a corresponding like-named component in any other figure.

Methods and systems are disclosed to automatically organize geological data and geochemical data of oil samples such that geological data may be interpreted from geochemical data of new oil samples. Traditionally, a geochemist may interpret geological data from geochemical data by manually organizing the geochemical data in the form of multi-dimensional plots, such as two-dimensional plots and star diagrams. However, manual organization and interpretation of the geochemical data by a geochemist may be time consuming. Thus, the disclosed methods and systems may be considered an improvement over the existing manual process traditionally used to determine geological data from geochemical data.

Returning to the present disclosure, previously collected geochemical data and geological data from oil samples may be automatically organized and extracted from a database using an artificial intelligence (AI) algorithm. The geological data may include organofacies data, depositional environment data, and oil family data. Further, the AI algorithm may flag missing geochemical data and geological data. The automatically organized and extracted geochemical data and geological data may be used to train a machine learning network to interpret or predict new geological data from new geochemical data for new oil samples. Routine geochemical analysis in a laboratory may then be conducted by a geochemist to determine new geochemical data for the new oil sample. The trained machine learning network may then be used to interpret or predict new geological data from the new geochemical data.

Turning to FIG. 1, FIG. 1 depicts a top-down view of a subterranean region of interest (100) in accordance with one or more embodiments. An oil field (102) may exist within the subterranean region of interest (100). The oil field (102) may contain one or more hydrocarbon reservoirs (104). One or more wells may penetrate each hydrocarbon reservoir (104) at a well location (106).

Each hydrocarbon reservoir (104) may have accumulated within the oil field (102) because specific geological requirements were met. For example, a convention hydrocarbon reservoir (104) may have formed due to the existence of a source rock, migration path, cap rock, reservoir rock, and trap.

Source rock is defined as rock rich in organic matter. Source rock may originate from various environments (hereinafter “origins”). Origins include, but are not limited to intrashelf/basinal, outer-inter shoal, shoal complex, slope/gravity flow, restricted lagoon, shallow intrashelf, tidal complex, mixed carbonate/clastics, and dolomite. Closely linked to the origin of source rock is “depositional environment” or the physical, chemical, and biological processes associated with the deposition of source rock. Depositional environments include, but are not limited to, marine, marine and marl, mixed marine and shale, fluvial deltaic, and lacustrine. In some embodiments, depositional environment may be directly linked to “organofacies.” Organofacies may be categorized into distinct categories that may be labeled A, B, C, or D. For example, organofacies A may be linked to marine, organofacies B may be linked to mixed marine and shale, organofacies C may be linked to fluvial deltaic, and organofacies D may be linked to lacustrine. In some embodiments, intermediate organofacies may additionally exist between the distinct categories.

Following the deposition of source rock, geologic time, heat, and overburden pressure, among other processes, may result in source rock ultimately generating hydrocarbons in the form of oil and natural gas. Hydrocarbons may be expelled from source rock to travel along a migration path. Various hydrocarbons may be denoted as being from the same “oil family” if the hydrocarbons were expelled from the same source rock.

The hydrocarbons may ultimately be stored in reservoir rock due to cap rock and a trap. The cap rock may present low permeability to stop hydrocarbons from flowing upwards to the surface. A trap, such as an anticline or pinch-out, may then keep the hydrocarbons in place. Note that hydrocarbons from different oil families may be stored in the same hydrocarbon reservoir (104).

Once hydrocarbons are expelled from a source rock, the hydrocarbons may undergo “alteration mechanisms.” Alteration mechanisms may include, but are not limited to, biodegradation, evaporation, water washing, weathering, and thermal alteration. Hereinafter, origin, depositional environment, organofacies, oil family, and alteration mechanism may be considered types of “geological data.”

Returning to FIG. 1, an oil field (102) may be in one or more of five phases in the oil field lifecycle: exploration, appraisal, development, production, and abandonment. If the oil field (102) is in the exploration, appraisal, development, or production phase, hydrocarbon oil samples (hereinafter “oil samples”) may be collected from wells that penetrate a hydrocarbon reservoir (104). In some embodiments, oil samples may be crude oil samples or refined oil samples.

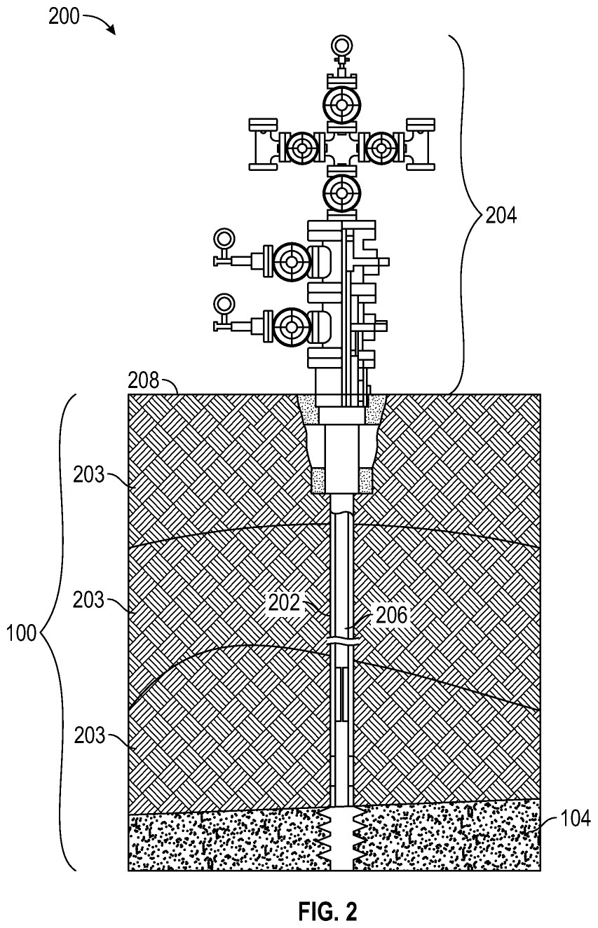

Oil samples may be extracted from a hydrocarbon reservoir (104) using a production system. FIG. 2 illustrates a production system (200) in accordance with one or more embodiments. Prior to using a production system (200), a well (202) may be drilled that traverses rock (203) within the subterranean region of interest (100) to ultimately penetrate the hydrocarbon reservoir (104). The production system (200) may include a wellhead (204) and production tubing (206) among other components (not shown). The production tubing (206) may be deployed downhole into the drilled well (202) to provide a flow path for hydrocarbon fluids to be transferred from the hydrocarbon reservoir (104) to the surface (208) and through the wellhead (204). The wellhead (204) may be further connected to a pipeline (not shown). The pipeline may be used to transfer the recovered hydrocarbon fluids from the well location (106) for midstream storage and downstream processing and usage. Crude oil samples may be extracted from the recovered hydrocarbon fluids prior to processing. Alternatively, refined oil samples may be extracted from the hydrocarbon fluids following processing.

In a laboratory, the oil samples may be analyzed for their geochemical properties. Geochemical properties (hereinafter “geochemical data”) include abundances of hydrocarbon molecules within the oil samples. Hydrocarbon molecules may be alkane series hydrocarbons, saturated cyclic hydrocarbons, aromatic hydrocarbons, and asphaltene hydrocarbons. Hydrocarbon molecules may also be categorized by their carbon number. For example, gas may range from approximately C₄ to C₇, light hydrocarbons may range from approximately C₇ to C₉, medium hydrocarbons may range from approximately C₁₀ to C₁₉, and heavy hydrocarbons may range from approximately C₂₀ to C₃₀. Further, some hydrocarbon molecules may be considered biomarkers.

In some embodiments, hydrocarbon molecules may be separated from an oil sample using gas chromatography (GC). In particular, GC with a flame ionization detector (FID) or gas chromatography-mass spectrometry (GC-MS) may be used. In GC, an oil sample may be volatized and passed through a chromatographic column using a carrier gas (often denoted the “mobile phase”). The chromatographic column may be a capillary column that includes a stationary phase. Further, the chromatographic column may reside in an oven to control the temperature of the carrier gas typically using a hold and ramp temperature profile. The carrier gas may be hydrogen. Individual or groups of hydrocarbon molecules within an oil sample will elute from the oil sample at different times due to having different affinities for the stationary phase. For example, as a carrier gas carries the oil sample through the chromatographic column, different hydrocarbon molecules will interact with the stationary phase for different lengths of time due to the different affinities. As such, different hydrocarbon molecules will travel at different velocities and thereby separate at different times. The time is takes for each hydrocarbon molecule to pass through the chromatographic column and separate is denoted “retention time.” Note that in other embodiments, GC may include a precolumn, which may be a packed column, where the oil sample passes through the precolumn prior to passing through the chromatographic column.

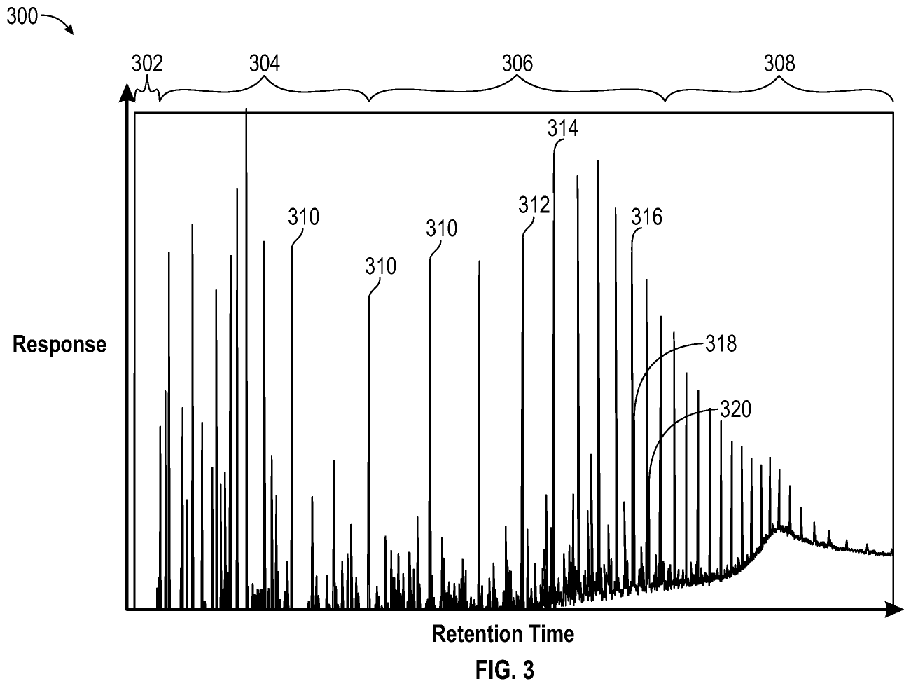

Following separation, individual or groups of hydrocarbon molecules are detected as a “response.” In some embodiments, the hydrocarbon molecules may be detected by an FID. Responses may be plotted relative to retention time to produce a chromatogram (300), as shown in FIG. 3. Each response may be associated to one or more specific hydrocarbon molecules based on when the specific hydrocarbon molecule(s) eluted from the oil sample.

The chromatogram (300) in FIG. 3 shows a series of responses from GC in accordance with one or more embodiments. The chromatogram (300) may generally identify gas (302), light hydrocarbons (304), medium hydrocarbons (306), and/or heavy hydrocarbons (308), some of which may be biomarkers. Each peak (310) on the chromatogram (300) may be identified by hydrocarbon number or as a specific hydrocarbon molecule, such as C₁₂ (312), C₁₃ (314), C₁₇ (316), pristane (318), and phytane (320). Each hydrocarbon number or specific hydrocarbon molecule may be identified using known standards that link retention time to hydrocarbon number or specific hydrocarbon molecule. The normalized abundance of identified hydrocarbon molecules may be determined using the area under each response, which may be determined by integration. Hereinafter, normalized abundances of identified hydrocarbon molecules are considered a type of geochemical data denoted “abundance data.”

In GC-MS, mass spectrometry is coupled to GC. GC-MS may allow for a finer degree of hydrocarbon molecule identification compared to GC alone. In GC-MS, the same process of GC as previously described may be used. The hydrocarbon molecules eluted during GC may then be detected and measured using a mass spectrometer. During mass spectrometry, each eluted hydrocarbon molecule, or group of eluted hydrocarbon molecules with the same retention time, is ionized typically using electron ionization where each hydrocarbon molecule is bombarded with a beam of free electrons emitted from a filament. Chemical ionization may alternatively be used. Hydrocarbon molecule-electron collisions may cause each hydrocarbon molecule to fragment into positively charged ions. The fragmented ions (hereinafter also “ions”) are then accelerated and subjected to an electric or magnetic field to cause deflection. Ions with the same mass-to-charge ratio will deflect by the same amount. The deflected ions may then be detected by an electron multiplier from which a mass spectrum may be displayed. A mass spectrum presents the relative abundance of ions detected by the electron multiplier relative to the mass-to-charge ratios of the ions. The mass spectrum may be referred to as a “fragmentation pattern” from which each hydrocarbon molecule of the oil sample may be identified by comparing the fragmentation pattern to a mass spectrum library. The mass-to-charge ratio may be represented as m/z were m is the mass of the ion and z is the number of elementary charges on the ion. Following GC-MS, each peak (310) on a chromatogram (300) may be identified as one or more hydrocarbon molecules based on when the hydrocarbon molecule(s) eluted during GC and what the mass-to-charge ratio determined during MS is. In some embodiments, MS may be isotope ratio mass spectrometry.

A person of ordinary skill in the art will appreciate that GC may be used to separate, measure, and identify tens of hydrocarbon molecules. Such hydrocarbon molecules may include, but are not limited to, dibenzothiophene, phenanthrene, pristane, phytane, toluene, 1,1-dimethylcyclopentane, 2-methylhexane, 3-methylhexane, 2,2-dimethylpentane, 2,3-dimethylpentane, 2,4-dimethylpentane, 3-ethylpentane, etc.

Following GC, ratios of the abundance of identified hydrocarbon molecules (hereinafter also “abundance ratios” or simply “ratios”) may be determined for an oil sample. Abundance ratios may include the comparison of the abundances of two hydrocarbon molecules, such as pristane/phytane. Ratios may also include the comparison of summations of hydrocarbon molecules such as 2,2-dimethylpentane/(3,2-dimethylpentane+2,3-dimethylpentane). A person of ordinary skill in the art will appreciate that tens to hundreds of other ratios may be used within the context of this disclosure. Hereinafter, abundance ratios are considered a type of geochemical data denoted “abundance ratio data.”

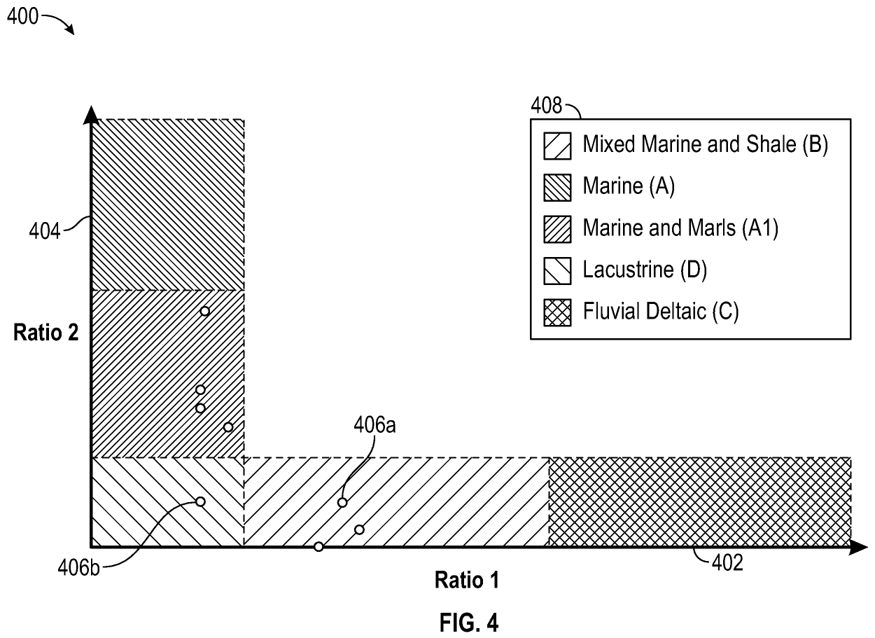

Traditionally, a geochemist may present useful abundance ratio data, known collectively to relate to geological data, on multi-dimensional plots such that the geochemist may manually identify geological data using the multi-dimensional plots. For example, FIG. 4 shows a two-dimensional plot (400) of two ratios in accordance with one or more embodiments. Ratio 1, shown on the abscissa (402), may be dibenzothiophene/phenanthrene. Ratio 2, shown on the ordinate (404), may be pristane/phytane. Ratio 1 and ratio 2 may be known by the geochemist to collectively relate to geological data, specifically, depositional environment data and organofacies data. Each point (406a, b) on the two-dimensional plot (400) is associated to one oil sample. Thus, the location of point 1 (406a) may be interpreted by the geochemist to indicate that the depositional environment of the oil sample is mixed marl and shale and the organofacies of the oil sample is B as shown by the key (408). The location of point 2 (406b) may be interpreted by the geochemist to indicate that the depositional environment of the oil sample is lacustrine and the organofacies of the oil sample is D.

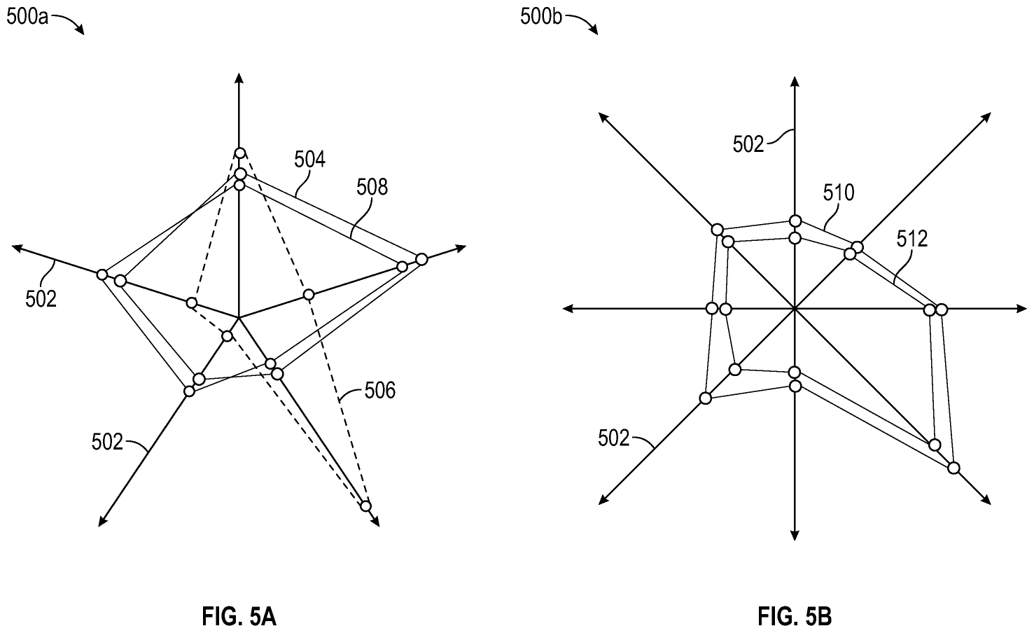

Traditionally, a geochemist may also present useful abundance ratio data, also known collectively to relate to geological data, on higher-dimensional plots, such as those shown in FIGS. 5A and 5B. FIGS. 5A and 5B may be referred to as “star diagrams” (500a, b) where each axis (502) represents a ratio and each polygon is associated to an oil sample. In some embodiments, each star diagram (500a, b) may be associated with ratios of hydrocarbon molecules of a particular carbon number, such as C₇. In some embodiments, the star diagram (500a) in FIG. 5A may be specifically referred to as a “correlation star diagram.” A correlation star diagram may be used by a geochemist to identify the oil family of an oil sample. For example, assume the oil family of hexagon 1 (504) and hexagon 2 (506) are known and of different oil families because the hexagon shapes are different. The oil sample used to determine hexagon 3 (508) may then be interpreted as belonging to the same oil family as the oil sample used to determine hexagon 1 (504) because the shape of hexagon 1 (504) and hexagon 3 (508) are similar.

Turning to FIG. 5B, the star diagram (500b) may be specifically referred to as a “transformation star diagram.” A transformation star diagram may be used by a geochemist to identify the alteration mechanism of an oil sample. For example, assume the oil sample used to determine octagon 1 (510) and the oil sample used to determine octagon 2 (512) belong to the same oil family. Further assume the eight ratios are known to relate to the alteration mechanism of biodegradation. Thus, the oil sample used to determine octagon 2 (512) may be interpreted as having biodegraded slightly relative to the oil sample used to determined octagon 1 (510) because the ratios of octagon 2 (512) are slightly smaller than the ratios of octagon 1 (510). In other embodiments, a transformation star diagram may be used to identify other alteration mechanisms through differences in one or more ratios or through one or more comparisons of ratios plotted on a transformation star diagram. In still other embodiments, the star diagram (500b) in FIG. 5B may be a correlation star diagram used to identify the oil family of an oil sample. A person of ordinary skill in the art will appreciate that other ratios and other multi-dimensional plots may be used to determine geological data about an oil sample.

To summarize, a vast amount of geochemical data may be determined for each oil sample following GC. Tens of hydrocarbon molecules may be identified by GS for each oil sample. Further, tens to hundreds of ratios may be determined for each oil sample. Useful geochemical data may then be presented on multi-dimensional plots as shown in FIGS. 4, 5A, and 5B. However, it may be time consuming for a geochemist to manually determine, organize, and plot useful geochemical data. Further, it may be time consuming for a geochemist to interpret the geochemical data to determine geological data of an oil sample. As such, this disclosure aims to reduce the time burden of a geochemist by automatically organizing geochemical data and geological data and automatically interpreting geochemical data to determine geological data. Thus, this disclosure is an improvement over the existing manual process performed by a geochemist to determine geological data from geochemical data.

In the context of this disclosure, an artificial intelligence (AI) algorithm may be used to automatically classify previously collected data as geochemical data, geological data, or other data. If data is classified as geochemical data, the AI algorithm may further classify the data as abundance data or abundance ratio data. If the data is classified as geological data, the AI algorithm may further classify the data as origin data, depositional environment data, organofacies data, oil family data, or alteration mechanism data. As such, the AI algorithm may be thought of as making one or more decisions about the data. Further, the AI algorithm may be considered a data mining approach. Hereinafter, “classify” and “categorize” will be considered synonymous and used interchangeably.

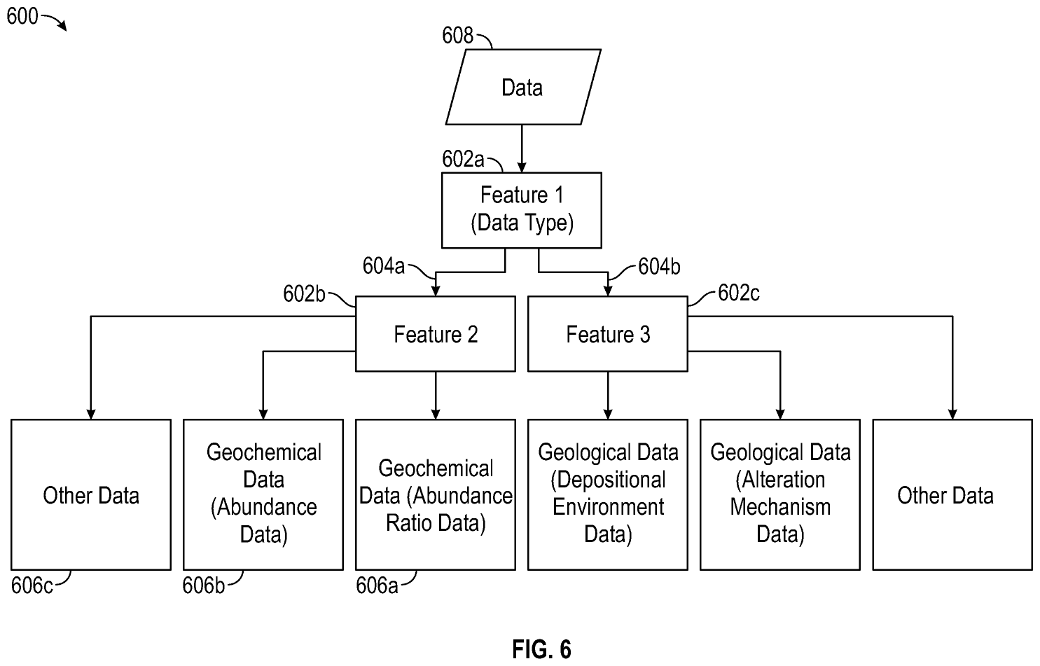

In some embodiments, a random forest algorithm (hereinafter also “random forest”) may be used to classify previously collected data. A random forest algorithm may consist of multiple decision trees. FIG. 6 illustrates a simplified decision tree (600) in accordance with one or more embodiments. A decision tree (600) may include nodes (602a-c), branches (604a, b), and leaves (606a-c). Each feature used to classify the previously collected data may be associated to a node (602a-c). FIG. 6 generally denotes a feature as feature 1, feature 2, or feature 3. Each feature may have multiple categories. Each category of a feature may be associated to a branch (604a, b). For example, a feature may be “gender” for which the categories of gender include “male” and “female.” The data (608) may ultimately be categorized by the decision tree (600) as specific geochemical data, geological data, or other data, each of which may be associated to a leaf (606a-c).

Once the decision tree (600) is constructed, the data (608) may be input into the decision tree (600) to classify the data (608) by feature to ultimately determine if the data (608) is specific geological data, geochemical data, or other data. For example, assume feature 1 is “data type” where the data type may be “numerical” or “categorical” as designated by branch 1a (604a) and branch 1b (604b), respectively. Depending on how the data (608) is classified by feature 1 determines what feature, feature 2 or feature 3, the data (608) is further classified by. For example, assume the data (608) is first classified as numerical. The data (608) will then be further classified by feature 2. Continuing with the example, feature 2 may be threshold values, such as threshold 1 and threshold 2. If the data (608) is below threshold 1, the data (608) may be classified as geochemical data (specifically, abundance ratio data) as shown by leaf 1a (606a). If the data (608) is between threshold 1 and threshold 2, the data (608) may be categorized as geochemical data (specifically, abundance data) as shown by leaf 1b (606b). If the data (608) is above threshold 2, the data (608) may be categorized as other data as shown by leaf 1c (606c).

To construct a random forest algorithm, multiple decision trees (600) may be constructed independent of one another. Each decision tree (600) may include some of the same features or different features relative to other decision trees (600). Following the construction of the decision trees (600) to create a random forest, the data is input into each decision tree (600). Each decision tree (600) will decide if the data (608) is specific geological data, geochemical data, or other data. The majority decision for all decision trees (600) ultimately determines if the data (608) is categorized as specific geological data, geochemical data, or other data. For example, if a random forest includes five decision trees (600) and three of those decision trees (600) categorize the data (608) as geological data (specifically, origin data), the data (608) will be categorized as origin data.

A person of ordinary skill in the art will appreciate that tens of features may be used to construct each decision tree (600). Further, a person of ordinary skill in the art will appreciate that AI algorithms other than a random forest algorithm may be used to categorize data (608) as specific geological data, geochemical data, or other data. In some embodiments, an artificial intelligence (AI) algorithm may further include a k-nearest neighbors algorithm. In some embodiments, a k-nearest neighbors algorithm may be used to identify missing geochemical data and geological data. Further still, in other embodiments, the AI algorithm may further include a least absolute shrinkage and selection operator (LASSO) algorithm and/or a k-means clustering algorithm. A person of ordinary skill in the art will appreciate that the AI algorithm may consist of numerous algorithms each of which may be used to perform different and/or similar tasks associated with ultimately categorizing data as specific geological data, geochemical data, or other data.

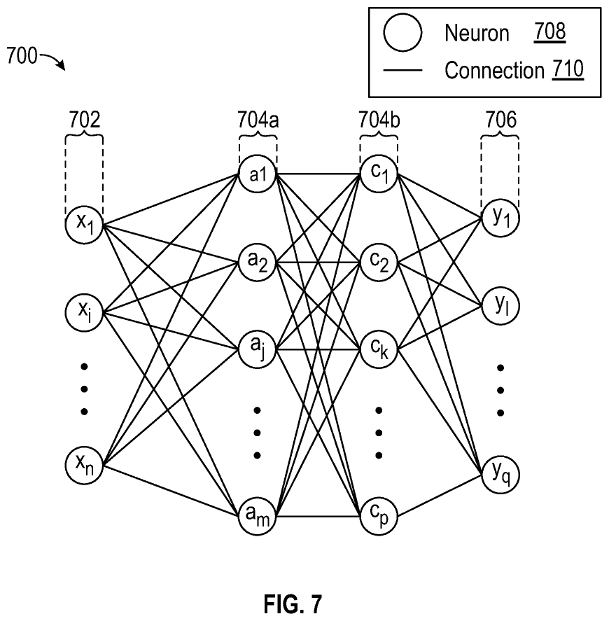

The geochemical data and geological data categorized using the AI algorithm may then be extracted and used as training data to train a machine learning (ML) network. In some embodiments, the ML network may be a neural network (700) as depicted in FIG. 7 in accordance with one or more embodiments. Note that machine learning is a subclass of AI. Machine learning may use algorithms and/or networks to predict data from other data. In the context of this disclosure, a neural network (700) may use a series of mathematical operations to predict geological data from geochemical data. A neural network (700) may include an input layer (702), one or more hidden layers (704a-b), and an output layer (706). The input layer (702) may receive geochemical data and the output layer (706) may present predicted geological data. The neural network (700) in FIG. 7 may be further described as a deep neural network (700) because multiple hidden layers (704a-b) exist. Further still, FIG. 7 may illustrate a standard feedforward deep neural network (700).

Each layer within a neural network (700) may represent an array. Further, each node or artificial neuron (708; hereinafter “neuron”) within a layer may represent an element within the array. A neuron (708) is loosely based on a biological neuron of the human brain. In FIG. 7, each neuron (708) within the input layer (702) may represent the element xᵢ within the array x, each neuron (708) within the first hidden layer (704a) may represent the element aⱼ within the array a, each neuron (708) within the second hidden layer (704b) may represent the element cₖ within the array c, and each neuron (708) within the output layer (706) may represent the element yₗ within the array y. Further to FIG. 7, each array is depicted as a one-dimensional array or vector. In other embodiments, each array may be any dimension.

One or more neurons (708) in one layer may be connected to one or more neurons (708) in neighboring layers through edges or connections (710). A connection (710) is loosely based on a synapse of the human brain. In some embodiments, a connection (710) may have a weight associated to it. For example, assume the input layer (702) and first hidden layer (704a) are “fully connected” or “densely connected.” In other words, assume all neurons (708) within the input layer (702) are connected to all neurons (708) within the first hidden layer (704a). Then, the weights for the connections (710) between the input layer (702) and the first hidden layer (704a) may make up an array of weights w⁽¹⁾ with elements wᵢⱼ where:

$${w}^{( 1 )} = [ \begin{matrix}{w}_{11} & {w}_{12} & {w}_{1 j} & … & {w}_{1 m} \\ {w}_{i 1} & {w}_{i 2} & {w}_{ij} & … & {w}_{im} \\ … & … & … & … & … \\ {w}_{n 1} & {w}_{n 2} & {w}_{nj} & … & {w}_{nm}\end{matrix} ] . \qquad Equation ( 1 )$$

In Equation (1), n is the total number of elements within the array x or the total number of neurons (708) within the input layer (702). Further, m is the total number of elements within the array a or the total number of neurons (708) within the first hidden layer (704a). The elements wᵢⱼ in each column of w⁽¹⁾ are the weights associated with the connections (710) between each of the elements xᵢ in the array x that connect to the same element aⱼ in the array a.

The value of each element aⱼ in the array a for the first hidden layer (704a) may be determined by:

aⱼ=gⱼ(bⱼ+Σᵢxᵢwᵢⱼ). Equation (3)

In Equation (3), the elements bⱼ of array b represent biases and the elements gⱼ of array g represent activation functions. In some implementations, the biases may be incorporated into the array of weights such that Equation (3) may be written as a₁=gⱼ(Σᵢxᵢwᵢⱼ). Each weight wᵢⱼ within the array of weights w⁽¹⁾ may amplify or reduce the significance of each element xᵢ relative to each element a₁. Activation functions gⱼ may include, without limitation, the linear function gⱼ(x)=x, the sigmoid function

$${g}_{j} ( x ) = \frac{1}{1 + {e}^{- x}} ,$$

the rectified linear unit (ReLU) function gⱼ(x)=max(0,x), and the scaled exponential linear unit (SeLU) function gⱼ(x)=Ax if x≥0 and gⱼ(x)=λα(e^{x}−1) if x<0, where λ and α are constants. A person of ordinary skill in the art will appreciate that other activation functions may also be used.

The connections (710) between the first hidden layer (704a) and the second hidden layer (704b) may make up another array of weights w⁽²⁾ with elements wⱼₖ. Equation (3) may be modified to determine the elements Cₖ of array c such that cₖ=gₖ(bₖ+Σⱼaⱼwⱼₖ). This process may be repeated until the elements yₗ within the array y are determined for the output layer (706). In summary, FIG. 7 may illustrate a standard feedforward deep neural network (700) that uses the mathematical operation of matrix multiplication, as presented in Equation (3), to predict the array y represented by the output layer (706) from the observed array x represented by the input layer (702). Note that FIG. 7 depicts a highly simplified and generic neural network (700).

Mathematical operations other than or in addition to matrix multiplication may be used within the architecture of a neural network (700). Other mathematical operations may include, but are not limited to convolution, concatenation, activation, pooling, batch normalization, and dropout.

Another type of neural network (700) that uses the mathematical operation of convolution, in additional to other mathematical operations, is a convolutional neural network (CNN).

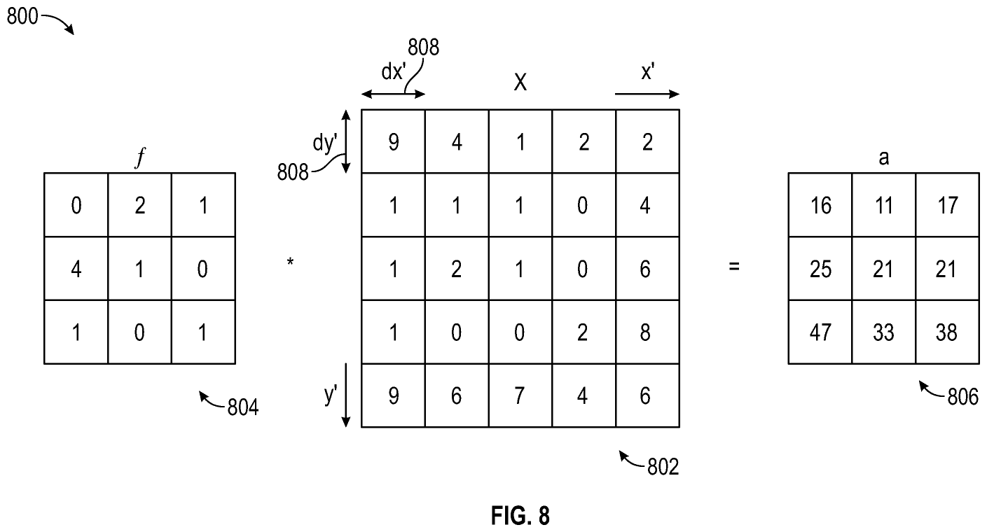

FIG. 8 illustrates convolution (800) in accordance with one or more embodiments. In FIG. 8, a square 3×3 convolution filter or kernel f (804) is convolved with a two-dimensional array x (802) to determine the array a (806) where:

a(x′,y′)=f*x(x′,y′)=Σ_{dx′=-a}^{a}Σ_{dy′=-b}^{b}f(dx′,dy′)×(x′−dx′,y′−dy′). Equation (4)

Here, the kernel f (804) contains the weights. The weights take values between 1 and 4 in FIG. 8. The operator “*” denotes convolution (800). Pairs (x′,y′) refer to the position within the array x (802). The step size dx′ and dy′ represent the stride (808), which both take a value of 1 in FIG. 8. The stride (808) is a hyperparameter of a CNN. In other embodiments, the array x (802) may be any dimensionality, the kernel f (804) may be rectangular and a different size, and the stride (808) may be an integer other than 1. The resulting array a (806) is denoted a “feature map” or “activation map.”

To convolve the kernel f (804) with the array x (802) using a stride (808) of 1, imagine the kernel f (804) sliding or translating along the array x (802) in increments or strides (808) of 1. A stride (808) of 1 for dx′ is one column within the array x (802) and a stride of 1 for dy′ is one row within the array x (802). For each translation of f along x, a linear combination of f and the portion of x that f is overlapping with determines one element of the array a (806) as described mathematically by Equation (4). For example, the first element of the array a (806; i.e., 16) is determined using the kernel f (804) and the first sub-array of the array x (802) that f overlaps with (i.e., [9 4 1; 1 1 1; 1 2 1]) such that:

(9·0)+(4·2)+(1·1)+(1·4)+(1·1)+(1·0)+(1·1)+(20)+(1·1)=16.

The kernel f (804) then translates to the right by a stride (808) of 1 in the x′ direction and the same calculation is performed to determine the second element of the array a (806; i.e., 11) where:

(4·0)+(1·2)+(2·1)+(1·4)+(1·1)+(0·0)+(2·1)+(1·0)+(0·1)=11.

The kernel f (804) may continue to slide by one column or one row at a time until all elements of the array a (806) are determined. As seen in FIG. 8, convolution (800) inherently down samples. To avoid down sampling, zero padding may be implemented around the array x (802) prior to convolution (800).

In practice, a CNN will convolve one or more kernels (804) with one section of an array x, one or more kernels (804) with another section of the array x, etcetera. This idea is known as “local connectivity” where each section of the array x that one or more kernels (804) is convolved with is a “receptive field.” If K kernels (804) are convolved with each of L sections of the array x, K·L activation maps are determined. If more than one activation map is determined, concatenation or stacking of all activation maps may be performed to determine a complete output. The size, types, and number of kernels (804) are other hyperparameters within a CNN.

Another mathematical operation that may be used within a CNN is activation. In some embodiments, activation may be performed following convolution (800). Activation may apply an activation function g, such as SeLU, to each element within the array a. No weights are associated with activation in reference to a CNN.

Yet another mathematical operation that may be used within a CNN is pooling. Pooling is another hyperparameter of a CNN. Pooling may be used to reduce the size of an array. Average pooling and maximum pooling are common pooling types.

Still other mathematical operations that may be used within a CNN are batch normalization and dropout. Batch normalization may normalize an array such that the elements within the array are between [−1,1]. Whereas dropout is a mathematical operation associated with training a CNN. Training may be defined as the process of determining the values of the weights and bias such that a neural network (700) makes accurate predictions. Training may be performed iteratively, where each iteration is commonly denoted an “epoch.” Each epoch may use a subset of the training data and backpropagation. Training may also further include the use of regularization, such as principle component analysis (PCA). Prior to an epoch, connections (710) will be randomly dropped or removed between two neighboring layers based on a dropout probability p. Backpropagation may then be performed for the epoch. Following backpropagation, the dropped connections (710) are reconnected. Connections (710) may be randomly dropped based on the dropout probability for any number of epochs.

Any of the mathematical operations discussed above (i.e., matrix multiplication, convolution (800), concatenation, activation, pooling, batch normalization, and dropout), and others not discussed, may be used in any quantity and any order to build a CNN as long as convolution (800) is used at least once. For example, the architecture of a CNN may be convolution (800), activation, dropout, batch normalization, concatenation, and activation performed in series. Further, in some embodiments, machine learning networks other than a neural network (700) may be used to predict geological data from geochemical data.

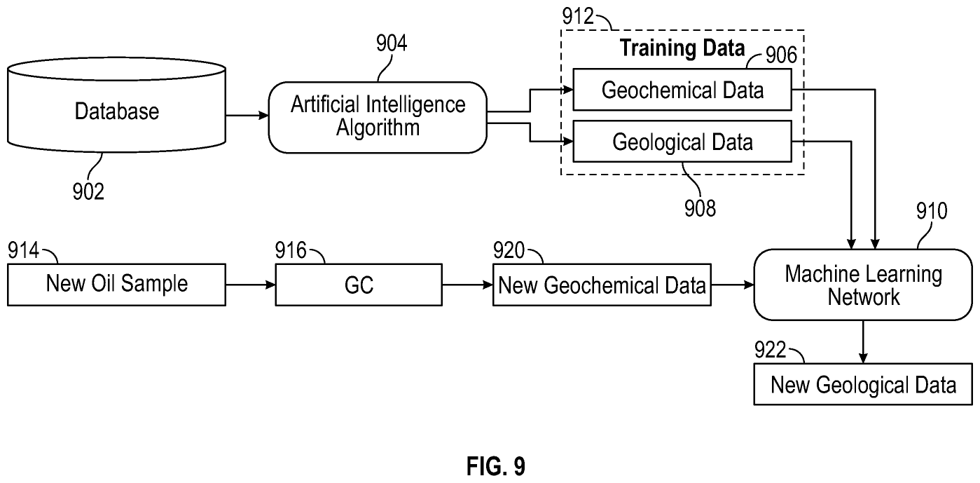

FIG. 9 shows a workflow in accordance with one or more embodiments. As previously described, a database (902) may store previously collected geochemical data and geological data associated to oil samples, among other data. In some embodiments, the geochemical data may have been previously determined using GC and manually organized and interpreted by a geochemist to determine geological data.

An AI algorithm (904) may access the database (902) to categorize the geochemical data (906) and geological data (908) that is stored in the database (902) among other data. In some embodiments, the AI algorithm (904) may separate the geochemical data (906) and geological data (908) from other data in the database (902). In some embodiments, the AI algorithm (904) may further separate the geological data (908) by origin data, depositional environment data, organofacies data, oil family data, and/or alteration mechanism data. In some embodiments, the AI algorithm (904) may additionally remove anomalous data and/or determine if any missing geochemical data (906) or geological data (908) is needed for adequate training of a ML network (910). The AI algorithm (904) may be, but is not limited to, a random forest algorithm (that includes decision trees (600) as described in FIG. 6), a k-nearest neighbors algorithm, a LASSO algorithm, and/or a k-means clustering algorithm.

The categorized geochemical data (906) and geological data (908) may then be used as training data (912) to train a ML network (910). The ML network (910) may be a neural network (700) or CNN, as previously described relative to FIGS. 7 and 8. The ML network (910) may alternatively be any other machine learning network familiar to a person of ordinary skill in the art.

Following training, a new oil sample (914) may be collected from the oil field (102) and analyzed using GC (916) to determine new geochemical data (920). The new geochemical data (920) may then be input into the trained ML network (910) to predict new geological data (922) for the new oil sample (914).

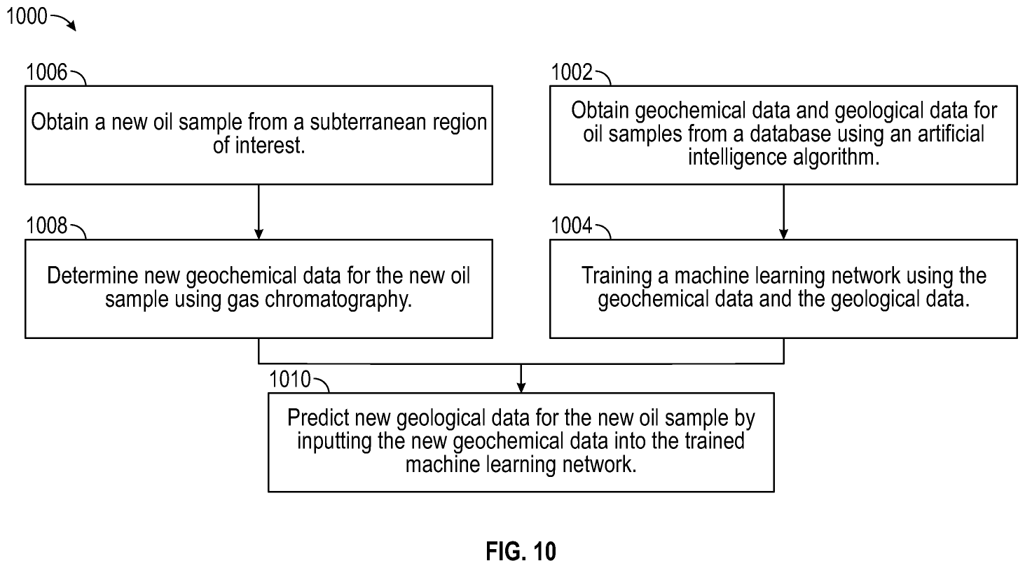

FIG. 10 shows a flowchart (1000) in accordance with one or more embodiments. In step 1002, previously collected geochemical data (906) and interpreted geological data (908) are obtained for oil samples. In some embodiments, the geochemical data (906) and the geological data (908) reside in a database (902) among other data. Further, in some embodiments, the geochemical data (906) and the geological data (908) may be categorized using an AI algorithm (904). In some embodiments, the AI algorithm (904) may additionally determine if geochemical data (906) and geological data (908) are missing and give an alert. In some embodiments, the AI algorithm (904) may additionally determine if geochemical data (906) and geological data (908) are outliers.

As previously described, the oil samples contain hydrocarbon molecules. The hydrocarbon molecules may range from gas to heavy hydrocarbons based on carbon number. In some embodiments, the carbon number may range from approximately C₄ to C₃₀. Specifically, hydrocarbon molecules may include pristane, toluene, and 1,1-dimethylcyclopentane among tens of other hydrocarbon molecules.

The geochemical data (906) contains abundances of the hydrocarbon molecules from the oil samples. In some embodiments, the geochemical data (906) may contain ratios of the abundances of hydrocarbon molecules. In some embodiments, the abundance data and/or the abundance ratio data may be presented on multi-dimensional plots. FIGS. 4, 5A, and 5B show examples of ratios presented on two-, five-, and eight-dimensional plots. In some embodiments, some multi-dimensional plots may be referred to as star diagrams (500a, b). The geological data (908) may include origin data, depositional environment data, organofacies data, oil family data, and/or alteration mechanism data.

In step 1004, the geochemical data (906) and the geological data (908) from the database (902) are used as training data (912) to train a ML network (910). In some embodiments, the ML network (910) may be a neural network (700), such as a CNN, as previously described in FIGS. 7 and 8. The training data (912) may be used to train the weights associated with the neural network (700) during backpropagation performed for numerous epochs.

In step 1006, a new oil sample (914) is obtained from a subterranean region of interest (100). The new oil sample (914) contains hydrocarbon molecules. In some embodiments, the new oil sample (914) is from the same oil field (102) or a neighboring oil field (102) as the oil samples from step 1002.

In step 1008, new geochemical data (920) is determined for the new oil sample (914). New geochemical data (920) is determined using GC (916). Like the geochemical data (906), the new geochemical data (920) may contain abundances of the hydrocarbon molecules from the new oil sample (914) in the form of abundance data and/or abundance ratio data. In some embodiments, the abundance data and/or abundance ratio data may be presented on multi-dimensional plots as described in FIGS. 4, 5A, and 5B.

In step 1010, the new geochemical data (920) is input into the trained ML network (910) to predict new geological data (922) for the new oil sample (914). Like the geological data (908), the new geological data (922) may include origin data, depositional environment data, organofacies data, oil family data, and/or alteration mechanism data associated with the new oil sample (914).

The flowchart (1000) presented in FIG. 10 may reduce the time consumption of a geochemist to determine geological data from geochemical data for a new oil sample. The process described in flowchart (1000) is an improvement to the existing manual and time-consuming process of determining geological data from geochemical data, and may also reduce error by removing the need for manual interpretation by a geochemist.

In some embodiments, the flowchart (1000) described in FIG. 10 may be repeated for multiple new oil samples (914). In these embodiments, a geological map (1100) may be generated using the new geological data (922) predicted by the trained ML network (910) for the multiple new oil samples (914).

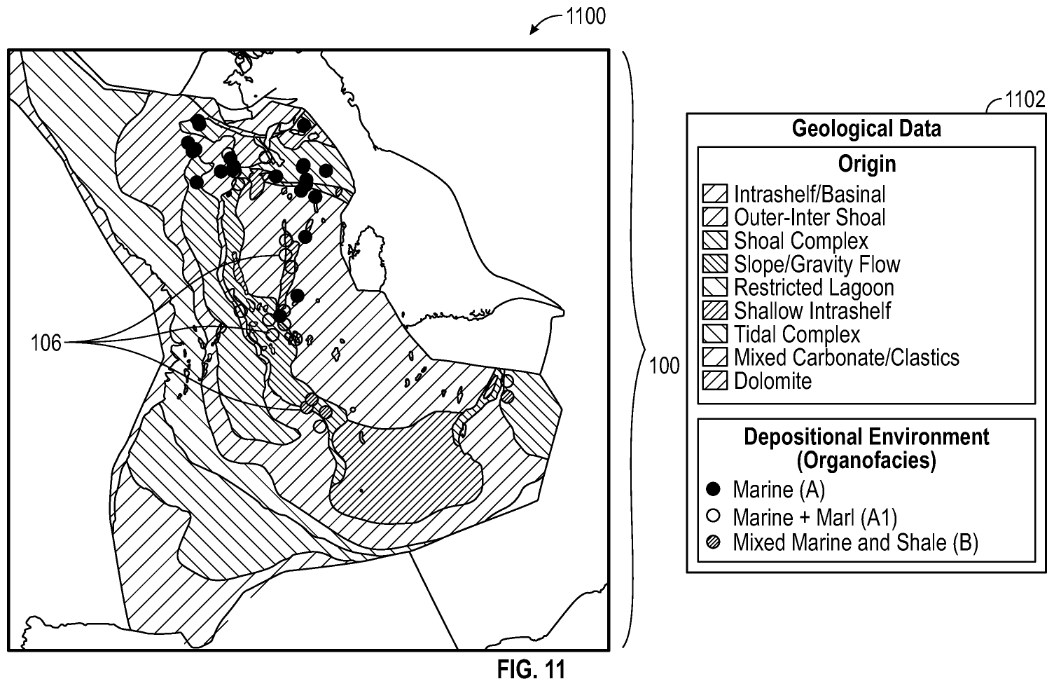

FIG. 11 shows a geological map (1100) in accordance with one or more embodiments. The geological map (1100) may be determined for a subterranean region of interest (100). The subterranean region of interest (100) may contain one or more hydrocarbon reservoirs (104) within one or more oil fields (102). One or more wells may access each hydrocarbon reservoir (104) as shown by each well location (106). Oil samples, including new oil samples (914), may be obtained at the well locations (106) using a production system (200). The new geological data (922) determined for the new oil samples (914) using the flowchart (1000) presented in FIG. 10 may be used to build the geological map (1100). For example, FIG. 11 specifically presents the new geological data (922) separated by origin data, depositional environment data, and organofacies data as shown by the key (1102).

The geological map (1100) may be used to determine an oil field management plan to further hydrocarbon recovery within one or more oil fields (102). The oil field management plan may use the geological map (1100) to identify possible migration paths and hydrocarbon reservoir compartmentalization. This information may then be used to plan when and where to drill new wells (202) to further access hydrocarbons within an oil field (102). The information may further be used to plan completion strategies for the newly drilled wells (202) in preparation for production, such as what casing to use and if hydraulic fractures should be induced. The information may further still be used to plan when, where, and how to stimulate current wells (202) to restore or enhance hydrocarbon recovery within an oil field (102).

The oil field management plan may also use the geological map (1100) to assess the location of well leaks. The oil field management plan may then use well leak information to determine where and how to stop leaks, such as by using a casing patch or by stopping production to replace casing.

Determining the production infrastructure, such as the size of the midstream and downstream facilities, may also be a part of the oil field management plan. As the oil field management plan progresses, the geological map (1100) may be updated to provide further insight into the current state of the oil fields (102) such that the oil field management plan may be updated to ensure the oil fields (102) are being adequately managed as the state of the oil fields (102) change.

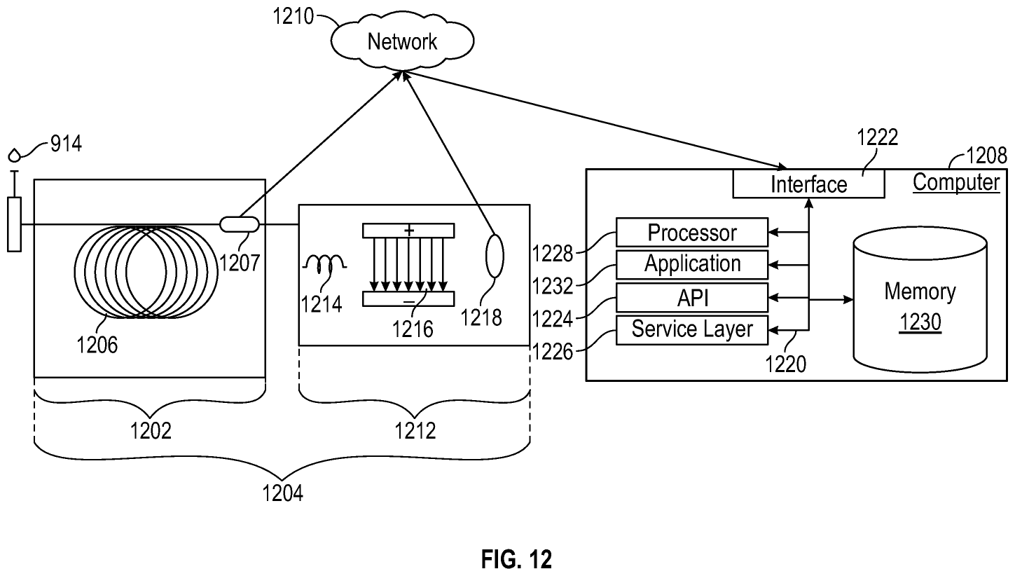

FIG. 12 depicts systems in accordance with one or more embodiments. A new oil sample (914) may be separated, measured, and identified by its hydrocarbon molecules using a GC-MS system (1204). The GC system (1202) may include a chromatographic column (1206) and an FID (1207). The chromatographic column (1206) may be a capillary column. In some embodiments, the GC system (1202) may include a precolumn (not shown). The chromatogram (300) output from the GC system (1202) may be transferred to a computer (1208) via a network (1210).

The GC-MS system (1204) may include a GC system (1202) coupled to a MS system (1212). The MS system (1212) may include a filament (1214) that fragments the separated hydrocarbon molecules into ions using a beam of free electrons. The ions are then subjected to an electric field (1216) and detected by an electron multiplier (1218). The mass spectrums and chromatogram (300) output from the GC-MS system (1204) may be transferred to a computer (1208) via a network (1210).

The computer (hereinafter also “computer system”) (1208) is used to provide computational functionalities associated with described AI algorithms (904), ML networks (910), other algorithms, methods, functions, processes, flows, and procedures as described in this disclosure, according to one or more embodiments. The illustrated computer (1208) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (1208) may include a computer that includes an input device, such as a keypad, keyboard, touch screen, or other device that can accept user information, and an output device that conveys information associated with the operation of the computer (1208), including digital data, visual, or audio information (or a combination of information), or a GUI.

The computer (1208) can serve in a role as a client, network component, a server, a database or other persistency, or any other component (or a combination of roles) of a computer system for performing the subject matter described in the instant disclosure. The illustrated computer (1208) is communicably coupled with a network (1210). In some implementations, one or more components of the computer (1208) may be configured to operate within environments, including cloud-computing-based, local, global, or other environment (or a combination of environments).

At a high level, the computer (1208) is an electronic computing device operable to receive, transmit, process, store, or manage data and information associated with the described subject matter. According to some implementations, the computer (1208) may also include or be communicably coupled with an application server, e-mail server, web server, caching server, streaming data server, business intelligence (BI) server, or other server (or a combination of servers).

The computer (1208) can receive requests over network (1210) from a client application (for example, from the GC-MS system (1204)) and responding to the received requests by processing the said requests in an appropriate software application. In addition, requests may also be sent to the computer (1208) from internal users (for example, from a command console or by other appropriate access method), external or third-parties, other automated applications, as well as any other appropriate entities, individuals, systems, or computers.

Each of the components of the computer (1208) can communicate using a system bus (1220). In some implementations, any or all of the components of the computer (1208), both hardware or software (or a combination of hardware and software), may interface with each other or the interface (1222) (or a combination of both) over the system bus (1220) using an application programming interface (API) (1224) or a service layer (1226) (or a combination of the API (1224) and service layer (1226). The API (1224) may include specifications for routines, data structures, and object classes. The API (1224) may be either computer-language independent or dependent and refer to a complete interface, a single function, or even a set of APIs. The service layer (1226) provides software services to the computer (1208) or other components (whether or not illustrated) that are communicably coupled to the computer (1208). The functionality of the computer (1208) may be accessible for all service consumers using this service layer. Software services, such as those provided by the service layer (1226), provide reusable, defined business functionalities through a defined interface. For example, the interface may be software written in JAVA, C++, or other suitable language providing data in extensible markup language (XML) format or another suitable format. While illustrated as an integrated component of the computer (1208), alternative implementations may illustrate the API (1224) or the service layer (1226) as stand-alone components in relation to other components of the computer (1208) or other components (whether or not illustrated) that are communicably coupled to the computer (1208). Moreover, any or all parts of the API (1224) or the service layer (1226) may be implemented as child or sub-modules of another software module, enterprise application, or hardware module without departing from the scope of this disclosure.

The computer (1208) includes an interface (1222). Although illustrated as a single interface (1222) in FIG. 12, two or more interfaces (1222) may be used according to particular needs, desires, or particular implementations of the computer (1208). The interface (1222) is used by the computer (1208) for communicating with other systems in a distributed environment that are connected to the network (1210). Generally, the interface (1222) includes logic encoded in software or hardware (or a combination of software and hardware) and operable to communicate with the network (1210). More specifically, the interface (1222) may include software supporting one or more communication protocols associated with communications such that the network (1210) or interface's hardware is operable to communicate physical signals within and outside of the illustrated computer (1208).

The computer (1208) includes at least one computer processor (1228). Although illustrated as a single computer processor (1228) in FIG. 12, two or more processors may be used according to particular needs, desires, or particular implementations of the computer (1208). Generally, the computer processor (1228) executes instructions and manipulates data to perform the operations of the computer (1208) and any algorithms, methods, functions, processes, flows, and procedures as described in the instant disclosure.

The computer (1208) also includes a memory (1230) that holds data for the computer (1208) or other components (or a combination of both) that can be connected to the network (1210). For example, memory (1230) can be a database storing data consistent with this disclosure. Although illustrated as a single memory (1230) in FIG. 12, two or more memories may be used according to particular needs, desires, or particular implementations of the computer (1208) and the described functionality. While memory (1230) is illustrated as an integral component of the computer (1208), in alternative implementations, memory (1230) can be external to the computer (1208).

The application (1232) is an algorithmic software engine providing functionality according to particular needs, desires, or particular implementations of the computer (1208), particularly with respect to functionality described in this disclosure. For example, application (1232) can serve as one or more components, modules, applications, etc. Further, although illustrated as a single application (1232), the application (1232) may be implemented as multiple applications (1232) on the computer (1208). In addition, although illustrated as integral to the computer (1208), in alternative implementations, the application (1232) can be external to the computer (1208).

There may be any number of computers (1208) associated with, or external to, a computer system containing a computer (1208), wherein each computer (1208) communicates over network (1210). Further, the term “client,” “user,” and other appropriate terminology may be used interchangeably as appropriate without departing from the scope of this disclosure. Moreover, this disclosure contemplates that many users may use one computer (1208), or that one user may use multiple computers (1208).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method, comprising:
obtaining, by a computer processor, geochemical data and geological data for a plurality of oil samples, wherein each oil sample comprises hydrocarbon molecules, wherein the geochemical data comprises abundances of the hydrocarbon molecules;
training, by the computer processor, a machine learning network using the geochemical data and the geological data;
obtaining a new oil sample from a subterranean region of interest;
determining new geochemical data for the new oil sample using gas chromatography; and
predicting, by the computer processor, new geological data for the new oil sample by inputting the new geochemical data into the trained machine learning network.

2. The method of claim 1, further comprising:
obtaining a plurality of new oil samples from the subterranean region of interest;
for each new oil sample among the plurality of new oil samples:
determining the new geochemical data for the new oil sample using gas chromatography, and
predicting the new geological data for the new oil sample by inputting the new geochemical data into the trained machine learning network; and
generating a geological map of the subterranean region of interest using the new geological data for each of the plurality of new oil samples.

3. The method of claim 2, further comprising determining an oil field management plan using the geological map.

4. The method of claim 1, wherein obtaining the geochemical data and the geological data further comprises:
determining the geochemical data and the geological data from a database using an artificial intelligence algorithm; and
determining if the geochemical data and/or the geological data for each oil sample is missing using the artificial intelligence algorithm.

5. The method of claim 4, wherein the artificial intelligence algorithm comprises a random forest algorithm.

6. The method of claim 4, wherein obtaining the geochemical data and the geological data further comprises:
determining a geological data outlier using the artificial intelligence algorithm.

7. The method of claim 1, wherein the geochemical data comprise ratios of the abundances of the hydrocarbon molecules.

8. The method of claim 1, wherein the geochemical data comprise star diagrams.

9. The method of claim 1, wherein the hydrocarbon molecules range from gas to heavy hydrocarbons.

10. The method of claim 1, wherein the geological data comprise depositional environment.

11. The method of claim 1, wherein the machine learning network comprises a convolutional neural network.

12. A non-transitory computer readable medium storing instructions executable by a computer processor, the instructions comprising functionality for:
obtaining geochemical data and geological data for a plurality of oil samples, wherein each oil sample comprises hydrocarbon molecules, wherein the geochemical data comprises abundances of the hydrocarbon molecules;
training a machine learning network using the geochemical data and the geological data;
receiving new geochemical data for a new oil sample from a subterranean region of interest; and
predicting new geological data for the new oil sample by inputting the new geochemical data into the trained machine learning network.

13. The non-transitory computer readable medium of claim 12, the instructions further comprising functionality for:
for each new oil sample among a plurality of new oil samples from the subterranean region of interest:
receiving the new geochemical data for the new oil sample, and
predicting the new geological data for the new oil sample by inputting the new geochemical data into the trained machine learning network; and
generating a geological map of the subterranean region of interest using the new geological data for the plurality of new oil samples.

14. The non-transitory computer readable medium of claim 12, wherein obtaining the geochemical data and the geological data further comprises:
determining the geochemical data and the geological data from a database using an artificial intelligence algorithm; and
determining if the geochemical data and/or the geological data for each oil sample among the plurality of oil samples is missing using the artificial intelligence algorithm.

15. A system, comprising:
a gas chromatography system configured to determine new geochemical data for a new oil sample; and
a computer processor configured to:
obtain geochemical data and geological data for a plurality of oil samples, wherein each oil sample comprises hydrocarbon molecules, wherein the geochemical data comprises abundances of the hydrocarbon molecules,
train a machine learning network using the geochemical data and the geological data,
receive the new geochemical data for the new oil sample, and
predict new geological data for the new oil sample by inputting the new geochemical data into the trained machine learning network.

16. The system of claim 15, further comprising a production system configured to extract the new oil sample from a well.

17. The system of claim 15, wherein the gas chromatography system and the computer processor are communicably coupled.

18. The system of claim 15, wherein the gas chromatography system comprises a chromatographic column.

19. The system of claim 15, wherein the gas chromatography system comprises a flame ionization detector.

20. The system of claim 15, wherein the gas chromatography system comprises mass spectrometry.
